(19) United States
(12) Patent Application Publication
(10) Pub. No.: US 2015/0283296 A1
(43) Pub. Date: Oct. 8, 2015

(54) BIOACTIVE GLASS WITH ETHYLENE OXIDE PROPLYENE OXIDE BLOCK COPOLYMERS

(71) Applicant: NOVABONE PRODUCTS LLC, Alachua, FL (US)

(72) Inventors: Cecilia A. Cao, Gainesville, FL (US); Gregory J. Pomrink, Newberry, FL (US)

(21) Appl. No.: 14/369,119

(22) PCT Filed: Dec. 17, 2013
(86) PCT No.: PCT/US13/75741

§ 371 Date: Jun. 26, 2014

Related U.S. Application Data
(60) Provisional application No. 61/738,585, filed on Dec. 18, 2012.
(60) Provisional application No. 61/787,827, filed on Mar. 15, 2013.

(51) Int. Cl. A61L 27/10; A61L 27/54; A61L 27/18

(52) U.S. Cl. CPC A61L 27/10; A61L 27/18; A61L 27/54; A61L 2300/112; A61L 2430/02; A61L 2300/10

(57) ABSTRACT

Bone repair composition including a bone repair material suspended in a mixture of non-random poly(oxyalkylene) block copolymer and water exhibiting reverse phase behavior when the temperature increases from room temperature to body temperature. There are many materials used today for the repair and regeneration of bone defects. Bone is a composite of collagen, cells, calcium hydroxyapatite crystals, and small quantities of other proteins of organic molecules that has unique properties of high strength, rigidity, and ability to adapt to varying loads.

### CROSS-REFERENCE TO RELATED APPLICATIONS

This application claims the benefit of U.S. Provisional Patent Applications No. 61/787,827, filed Mar. 15, 2013, and No. 61/738,585, filed Dec. 18, 2012, the entire contents of which are hereby incorporated herein by reference.

### BACKGROUND OF THE INVENTION

There are many materials used today for the repair and regeneration of bone defects. Bone is a composite of collagen, cells, calcium hydroxyapatite crystals, and small quantities of other proteins of organic molecules that has unique properties of high strength, rigidity, and ability to adapt to varying loads. When bone injuries occur, it is necessary to fill voids or gaps in the bone as well as to encourage the repair and regeneration of bone tissue. One material useful to encourage such repair and regeneration is bioactive glass.

Bioactive glass was originally developed in 1969 by L. Hench. Additionally bioactive glasses were developed as bone replacement materials, with studies showing that bioactive glass can induce or aid in osteogenesis. Hench et al, J. Biomed. Mater. Res. 5:117-141 (1971). Bioactive glass can form strong and stable bonds with bone. Piotrowski et al., J. Biomed. Mater. Res. 9:47-61 (1975). Further, bioactive glass is not considered toxic to bone or soft tissue from studies of in vitro and in vivo models. Wilson et al., J. Biomed. Mater. Res. 805-817 (1981). Exemplary bioactive glasses known in the art include 45S5, 45S5B1, 58S, and 570C30. The original bioactive glass, 45S5, is melt-derived. Sol-gel derived glasses have nanopores that allow for increased surface area and bioactivity.

There are drawbacks to the use of bioactive glass or other materials in the form of liquids, pastes, and solids to fill voids or gaps in the bone. A liquid or a paste may not remain at the site of the void or gap in the bone. A solid may be difficult to apply and may not conform well to the void or gap in the bone.

These drawbacks may be reduced and/or eliminated by adding materials to a bone repair composition such that the composition is a liquid at room temperature and more of a paste or solid at the temperature of the body. Such compositions are described in U.S. Pat. No. 6,623,748 to Clokie, which is incorporated by reference in its entirety herein.

### BRIEF SUMMARY OF THE INVENTION

The invention provides a bone repair composition comprising a bone repair material suspended in a mixture of poly(oxyalkylene) block copolymer and water exhibiting reverse phase behavior, i.e. becoming more viscous when the temperature increases from room temperature to body temperature. The bone repair material can be any number of materials that assist in bone repair and production. Such materials include at least bioactive glass, spherical bioactive glass in a bimodal size distribution, and tricalcium phosphate, i.e. silicated tricalcium phosphate. The present invention is further directed to bioactive glass particles with a polaxamer coating and a putty or paste including such polaxamer coated particles of bioactive glass.

### DETAILED DESCRIPTION OF THE INVENTION

The invention provides a bone repair composition comprising a bone repair material suspended in a mixture of poly(oxyalkylene) block copolymer and water exhibiting reverse phase behavior when the temperature increases from room temperature to body temperature. The bone repair material may be a solid that is suspended in the bone repair composition.

The bone repair composition has a unique physical property of being flowable at refrigerated temperatures and increasingly solidified at higher temperatures, such as ambient and body temperatures. This property is referred to as “reverse phase” or “reverse thermal behavior” because most forms of matter become progressively more flowable as temperature increases. The composition is initially manufactured at low temperatures, i.e. at 5° C., such that the components of the composition may be mixed and/or suspended most thoroughly. The composition may then be raised to a higher temperature so as to allow for the components to remain in suspension.

In some embodiments, the composition is substantially a liquid at 5° C. and substantially a solid at 37° C. This effect can arise from a reverse phase transition from a liquid at lower temperature to a solid at higher temperature. As the temperature rises, the composition becomes substantially more viscous to allow the bone repair material, for example to bioactive glass, to more readily remain at the defect site. Generally, the composition is twice as viscous at 35° C. as compared to 0° C.

The bone repair composition provides for an acceleration in the rate and an enhancement in the quality of newly-formed bone. Improved bone healing may occur in those who may be compromised, such as diabetics, smokers, the obese, the elderly, those who have osteoporosis, those who use steroids, and those who have infections or other diseases that reduce the rate of healing. The rapid hardening of the bone repair composition at the site of the bone defect can serve to localize the bone repair material, such as bioactive glass, at the site.

The bone repair composition may be provided to a site of a bone defect by means of a syringe or other injection device. The bone repair composition is sufficiently liquid so as to be injectable, yet can harden suitably at the bone defect site at body temperature. For instance, if the bone repair composition is a liquid at room temperature, it may become a thick gel at body temperature. Alternatively, it may be described that the bone repair composition cures upon application to a bone defect at body temperature.

The bone repair composition has the advantages of low viscosity, runny liquid compositions with regard to the ease of application to a bone defect site. The inventive composition also provides for the advantages of a more solid paste-like composition that remains positioned at the defect after being applied. The solidification of the composition at body temperature overcomes the disadvantageous property of other liquid compositions that do not exhibit reverse phase behavior to flow away from the bone defect at 37° C. At the same time, because the composition is not a solid at room temperature, there is greater ease of applying the composition, such as by means of a syringe. The composition need not be laboriously painted onto a bone defect or applied onto a bone defect by means of pressure.

Other delivery modes can be used for more viscous bone repair compositions. These modes include painting the gel or paste directly onto a bone defect or extruding the gel or paste as a bead. If the bone repair composition is a gel at room temperature, it may become a paste at body temperature. If the bone repair composition is a thick gel or paste at room temperature, it may become a putty or a solid at body temperature. The relative amount of poly(oxyalkylene) block copolymer in the composition will determine the viscosity at room temperature and at body temperature.

In various embodiments, the poly(oxyalkylene) block copolymer is a poloxamer. The poloxamer may be Poloxamer 407. The poly(oxyalkylene) block copolymer also is biocompatible, non-rigid, amorphous, and has no defined surfaces or three-dimensional structural features.

Poloxamers are triblock copolymers composed of PEO and PPO units in the following structure: PEO-PPO-PEO. A particularly useful poloxamer in the context of the invention is Poloxamer 407 (Pluronic® F127). Poloxamer 407 has a high ratio of PEO to PPO and a high molar mass as compared to other poloxamers. The viscosity increases considerably as the temperature increases from 5° C. to 37° C. At a temperature below 25° C., a 20 wt % Poloxamer 407 solution will behave like a viscous liquid while at body temperature (37° C.), the same solution will behave like a semi-solid gel. The reverse phase property can arise from the discrete blocks of both hydrophilic (oxyethylene) and hydrophobic (oxypropylene) subunits of Poloxamer 407. Non-random alkyene oxide copolymers, such as Poloxamer 407, have advantages when used with bioactive glass over random copoylmers. For example, non-random copolymers may be readily mixed in water to yield a thermoreversible composite whereas random copolymers alone cannot readily be formulated with water to yield a thermoreversible composite. The non-random poloxamers described herein may be formulated with bioactive glass and blood.

Poloxamer 407 is regarded as non-toxic. The biodegradability can be improved by using forms of Poloxamer 407 in which there are carbonate linkages incorporated into the structure.

The physical properties of Poloxamer 407 are extensively described in Li et al., “Thermoreversible micellization and gelation of a blend of Pluronic® polymers” Polymer 49 (2008):1952-1960, which is incorporated by reference in its entirety herein. The properties of Poloxamer 407 are also described in Lenaerts et al., “Temperature-dependent rheological behavior of Pluronic® F127 aqueous solutions” International Journal of Pharmaceutics, 39 (1987): 121-127, which is incorporated by reference in its entirety herein, and in Ivanova et al., “Effect of Pharmaceutically Acceptable Glycols on the Stability of Liquid Crystalline Gels Formed by Poloxamer 407 in Water” Journal of Colloid and Interface Science, 252 (2002): 226-235, which is incorporated by reference in its entirety herein.

The mixture of poloxamers with a bone growth factor material (i.e. BMP-2) is described by Rey-Rico et al., “Osteogenic efficiency of in situ gelling poloxamine systems with and without bone morphogenetic protein-2” European Cells and Materials 21 (2011):317-340, which is incorporated herein by reference in its entirety.

It is known in the art that other poloxamers may be used as well, provided that the poloxamers are substantially liquid at room temperature and have a higher viscosity at body temperature. Generally such poloxamers have a high PEO content, as described on page 227 of Ivanova et al. Poloxamer P105, described in Li et al., may be used. Also, Poloxamer 105 or any other poloxamer may be added to Poloxamer 407 or to any other polaxmer to obtain an optimal viscosities at both room temperature and body temperature. Further, Poloxamer 407 or any other poloxamer used may be modified with means of adding functional groups. The functaional groups may be hydroxyl end groups, for example. Also, functional groups may have a positive charge such that the modified poloxamer is cationic.

In some embodiments, the weight ratio of the poly(oxyalkylene) block copolymer is 10%-90% relative to the weight of the bone repair composition. This weight ratio may be from 10-20%, 20-30%, 30%-40%, 40%-50%, 50%-60%, 60%-70%, 70%-80%, or 80%-90%. Alternatively, this weight ratio may be about 10%, about 11%, about 12%, about 13%, about 14%, about 15%, about 16%, about 17%, about 18%, about 19%, about 20%, about 21%, about 22%, about 23%, about 24%, about 25%, about 26%, about 27%, about 28%, about 29%, about 30%, about 31%, about 32%, about 33%, about 34%, about 35%, about 36%, about 37%, about 38%, about 39%, about 40%, about 41%, about 42%, about 43%, about 44%, about 45%, about 46%, about 47%, about 48%, about 49%, about 50%, about 51%, about 52%, about 53%, about 54%, about 55%, about 56%, about 57%, about 58%, about 59%, about 60%, about 61%, about 62%, about 63%, about 64%, about 65%, about 66%, about 67%, about 68%, about 69%, about 70%, about 71%, about 72%, about 73%, about 74%, about 75%, about 76%, about 77%, about 78%, about 79%, about 80%, about 81%, about 82%, about 83%, about 84%, about 85%, about 86%, about 87%, about 88%, about 89%, or about 90%. The material may have the consistency of a gel, putty, or any other non-liquid substance at room temperature.

The compositions may vary in molecular weight and be blended in ratios of 10:1 to 1:10.

The compositions may further comprise ions and other compounds that may be dissolved in water. It is known in the art that the addition of salts, such as PBS, can enhance the reverse phase solidification and setting properties of poloxamers, such as Poloxamer 407. Divalent salts may be particularly useful to improve the rheological properties of compositions containing Poloxamer 407 and bioactive glass materials as well as those of compositions containing Poloxamer 407 and other solid bone repair materials. The presence of divalent salts and other salts, such as PBS, and solid bone repair materials, such as bioactive glass, may lead to a synergistic increase in the reverse phase behavior of the poloxamers, such as Poloxamer 407.

The bone repair material may be osteoinductive, osteoconductive, or a material that is both osteoinductive and osteoconductive. The bone repair material may be xenogeneic, allogeneic, autogeneic, and/or alloplastic. The bone repair material may also be any combination of various therapeutic materials.

In some embodiments, the bone repair material is bioactive glass. Bioactive glass used in the invention may be melt-derived or sol-gel derived. Depending on their composition, bioactive glasses of the invention may bind to soft tissues, hard tissues, or both soft and hard tissues. The composition of the bioactive glass may be adjusted to modulate the degree of bioactivity. Furthermore, borate may be added to bioactive glass to control the rate of degradation.

In some embodiments, the bioactive glass contains silica and/or boron as well as other ions such as sodium and calcium.

The various types of bioactive glass that may be used as bone repair material in the invention are described at least in paragraphs 17-44 of U.S. Provisional Patent Application No. 61/702,445, filed on Sep. 18, 2012, the entire contents of which is incorporated by reference herein. The silica and/or calcium ions released by the bioactive glass may improve the expression of osteostimulative genes. The silica and/or calcium ions may also increase the amount of and efficacy of proteins associated with such osteostimulative genes. In several embodiments of the invention, the bone repair material is osteostimulative and can bring about critical ion concentrations for the repair and regeneration of hard tissue without the necessity of any therapeutic materials or agents.

In some embodiments, the bone repair material is 45S5 bioactive glass. The 45S5 bioactive glass may vary in size from 1 micrometer to 5 millimeters. The bioactive glass may be about 1-5 micrometers, about 5-15 micrometers, about 15-50 micrometers, about 50-200 micrometers, about 200-1,000 micrometers, about 1-2 millimeters, about 2-3 millimeters, about 3-4 millimeters, or about 4-5 millimeters.

In some embodiments, the bone repair material is a composition comprising calcium salt and silica. The silica is in the form of an inorganic silicate that is adsorbed onto the surface of the calcium salt. The silica is not incorporated into the structure of the calcium salt. The composition may be bioactive. These and other bone repair materials are described in U.S. Provisional Patent Application No. 61/656,741, filed on Jun. 7, 2012, the entire contents of which is incorporated by reference herein.

In some embodiments, the bone repair material is a composition comprising suspended autograft bone particles and suspended bioactive glass particles. Similar bone repair materials are described in U.S. Provisional Patent Application No. 61/641,961, filed on May 3, 2012, the entire contents of which is incorporated by reference herein, and in U.S. Provisional Patent Application No. 61/623,357, filed on Apr. 12, 2012, the entire contents of which is incorporated by reference herein.

The suspended bioactive glass particle may comprise SiO₂. Alternatively, the suspended bioactive glass particle may comprise P₂O₅, PO₃. or PO₄. The suspended bioactive glass particle may comprise B₂O₃ as well. In some embodiments, the suspended bioactive glass particle may comprise 40-60% SiO₂, 10-20% CaO, 0-4% P₂O₅, and 19-30% NaO. The suspended bioactive glass particle may further comprise a carrier selected from the group consisting of hydroxyapatite and tricalcium phosphate.

In some embodiments, the bioactive glass particle has a diameter of between about 1 micrometer and about 2,000 micrometers. The autograft bone particle and the bioactive glass particle may be pretreated in a solution comprising one or more of blood, bone marrow aspirate, bone-morphogenetic proteins, platelet-rich plasma, and osteogenic proteins. In various embodiments, the bioactive glass particle may not include any substantial amount of polymer.

In some embodiments, the bone repair material is a bioactive glass coated with a glycosaminoglycan, in which the glycosaminoglycan is bound to the bioactive glass. This and other bone repair materials are described in U.S. Provisional Patent Application No. 61/702,445, filed on Sep. 18, 2012, the entire contents of which is incorporated by reference herein.

In some embodiments, the bone repair material is a bimodal bioactive glass composition comprising large bioactive glass particles and small bioactive glass particles. The large bioactive glass particles have a substantially spherical shape and a mean diameter of between about 90 micrometers and about 2,000 micrometers. The small bioactive glass particles have a substantially spherical shape and a mean diameter of between about 10 micrometers and about 500 micrometers.

In some embodiments, the bone repair material is a trimodal bioactive glass composition comprising large bioactive glass particles, medium bioactive glass particles, and small bioactive glass particles. The large bioactive glass particles have a substantially spherical shape and a mean diameter of between about 1,000 micrometers and about 2,000 micrometers. The medium bioactive glass particles have a substantially spherical shape and a mean diameter of between about 90 micrometers and about 710 micrometers. The small bioactive glass particles have a substantially spherical shape and a mean diameter of between about 32 micrometers and about 125 micrometers.

In any of the above embodiments, small bioactive glass fibers may be added to the bone repair material. The small bioactive glass fibers have a diameter of less than 2 millimeters. The small bioactive glass fibers may be present in up to 40% by weight relative to the total weight of the bioactive glass. In various embodiments, the weight ratio of small bioactive glass fibers to total weight of the bioactive glass may be from 0-10%, 0-5%, 5-10%, 5-15%, 10-15%, 10-20%, 15-20%, 15-25%, 20-25%, 20-30%, 25-30%, 25-35%, 30-35%, 30-40%, or 35-40%. The weight ratio of small bioactive glass fibers to total weight of the bioactive glass may be about 1%, 2%, 3%, 4%, 5%, 6%, 7%, 8%, 9%, 10%, 11%, 12%, 13%, 14%, 15%, 16%, 17%, 18%, 19%, 20%, 21%, 22%, 23%, 24%, 25%, 26%, 27%, 28%, 29%, 30%, 31%, 32%, 33%, 34%, 35%, 36%, 37%, 38%, 39%, or 40%.

In some embodiments, any subset of the bioactive glass present, such as bioactive glass particles and/or small bioactive glass fibers, may be coated with silane as described in Verne E et al., “Surface functionalization of bioactive glasses” J. Biomed. Mater. Res. A., 2009, 90(4) 981-92. The silane or other functional coatings may then allow for binding of proteins onto the bioactive glass, such as BMP-2.

In some embodiments, any subset of the bioactive glass present, such as bioactive glass particles and/or small bioactive glass fibers, may have additional silicate chains present on them. The additional silicate chains may allow the bioactive glass particles and fibers to interact with one another, as well as with the EO and PO groups on the poloxamers. The effect of these interactions may be to reduce the surface area of the filler, increase resin demand, and to allow for higher filler loadings.

In some embodiments, any subset of the bioactive glass present, such as bioactive glass particles and/or small bioactive glass fibers, may have added hydroxyl triethoxysilanes coated onto the glass. Some of these silanes are available from Gelest, Inc. For example, the glass may be coated with hydroxyl(polyethyleneoxy) propyltriethoxysilane. Additionally, the glass may be coated with other organic substituted ethoxy- and methoxy-silanes that are effective to create an interaction between the coated glass and the EO/PO carrier.

In any of the above embodiments, the bone repair composition may be applied by a syringe at ambient temperature. After application to the bone or other site within the body at 37° C., the bone repair composition will harden and have a substantially lower tendency to migrate away from the application site.

More viscous bone repair compositions may be applied by painting the composition onto a site at or near the bone defect. Alternatively, more viscous bone repair compositions may be extruded onto the site in the form of a bead.

The invention provides for a method for treating a bone having a bone defect comprising contacting the bone at or near the site of the bone defect with a bone repair composition of any of the above-described embodiments.

The invention also provides for a method for treating a bone having a bone defect comprising placing a bone repair composition of any one of the above-described embodiments at a site of a bone gap or a bone defect.

A bone defect includes bony structural disruptions in which repair is needed. A bone defect may be a void, which is understood to be a three-dimension defect that includes a gap, cavity, hole or other substantial disruption of the structural integrity of the bone or joint. Gaps may be at least 2.5 cm and are generally in the range of 3-4 cm. This size is large enough so that spontaneous repair is not likely to occur and/or be complete. Exemplary bone defects include tumor resection, fresh fractures, cranial and facial abnormalities, spinal fusions, and loss of bone from the pelvis.

The invention further provides for a method for treating a bone having a bone defect comprising placing a bone repair composition of any one of the above-described embodiments at a bone gap or a bone defect.

Any of the above-described materials or methods may be undertaken to treat any number of bone defects. The bone defects may be a gap in the bone or may arise from lack of adequate bone regeneration. The bone defects may also be holes or fractures. The bone defects may also arise in the context of oral bone defects. The different types of bone defects are apparent to those of ordinary skill in the art. The various embodiments of the invention may be particularly useful with respect to orthopedic and spine processes because the material will stabilize and hold a better structure as it becomes more solidified when it heats up to body temperature.

In some embodiments, any of the above-described materials or methods may be combined with autograft bone chips for placement onto or near a bone defect. The materials may be a liquid or a gel at room temperature with the autograft bone chips suspended therein. Upon placement at or near the bone defect, the material will solidify around the autograft bone chips and serve to prevent the autograft bone chips from migrating away from the surgical sites.

In some embodiments, any of the above-described materials or methods may be combined with particles containing allogeneic or xenogeneic bone mineral for placement onto or near a bone defect. The materials may be a liquid or a gel at room temperature with the particles suspended therein. Upon placement at a surgical site, which is at or near the bone defect, the material will solidify around the particles and serve to prevent the particles from migrating away from the surgical site.

In various embodiments of the invention, the bone repair material is entirely synthetic. Advantages of using such a bone repair material include the elimination of substantially all risk of disease transmission.

In various embodiments of the invention, the bone repair material is not a natural bone material or a synthetic bone material.

Any of the above-described aspects and embodiments of the invention may be in injectable form. Injection may occur by means of a syringe, for example. The compositions are particularly useful when injected in a gel or liquid form into a bone gap or bone defect. The injected gel or liquid would then solidify at body temperature when placed on or near the bone gap or the bone defect.

### Example 1

### Preparation of a Bone Repair Composition Gel

A solution is prepared by dissolving 25 weight percent of Pluronic® F127 powder in 75 weight percent sterile water at 5° C. The composition is then prepared by mixing 70 weight percent of the solution with 30 weight percent bioactive glass at 5° C. At 25° C., the composition has a gel-like consistency. In order to maintain proper suspension of the bioactive glass, the composition is placed on a rotating shaker so that the bioactive glass particles are suspended.

### Example 2

### Preparation of a Bone Repair Composition Paste

A solution is prepared by dissolving 25 weight percent of Pluronic® F127 powder in 75 weight percent sterile water at 5° C. The composition is then prepared by mixing 50 weight percent of the solution with 50 weight percent bioactive glass at 5° C. At 25° C., the composition has a paste-like consistency. If there is any concern that the bioactive glass is no longer uniformly suspended in the composition, the composition is cooled to 5° C., placed on a rotating shaker, and then gradually heated to 25° C.

### Example 3

### Treatment of Osteochondral Defects in Rabbits

A bone repair composition is prepared by suspending bioactive glass particles having a diameter of 20 microns in a 30 wt. % Poloxamer 407 aqueous solution. The composition is a liquid at room temperature.

A total of six adult rabbits are utilized. Two osteochondral defects are created bilaterally in the patellar sulcus of each animal using standard surgical techniques. The bone repair compositions are applied by syringe into the left osteochondral defect of each animal. The composition is liquid at room temperature and hardens quickly to a semi-solid paste consistency upon application to the osteochondral defect. The animals are then sutured and monitored for twelve weeks post-operation.

After twelve weeks, the animals are sacrificied and the defect sites are evaluated. In all of the animals, the treated osteochondral defect shows considerable healing with regeneration of both bone and cartilage.

### Example 4

### Comparative Treatment of Osteochondral Defects in Rabbits

A test bone repair composition is prepared by suspending bioactive glass particles having a diameter of 20 microns in a 30 wt. % Poloxamer 407 aqueous solution. The composition is a liquid at room temperature. A control bone repair composition is prepared by using the same bioactive glass particles in the form of a paste at room temperature, without use of any poloxamer to form the paste.

A total of six adult rabbits are utilized. Two osteochondral defects are created bilaterally in the patellar sulcus of each animal using standard surgical techniques. The test bone repair compositions are applied by syringe into the left osteochondral defect of each animal. The test composition is liquid at room temperature and hardens quickly to a semi-solid paste consistency upon application to the osteochondral defect. The control bone repair compositions are applied by extrusion into the right osteochondral defect of each animal. The animals are then sutured and monitored for twelve weeks post-operation.

After twelve weeks, the animals are sacrificed and the defect sites are evaluated. In all of the animals, the left osteochondral defect shows considerable healing with regeneration of both bone and cartilage. Comparatively, the right osteochondral defect shows a reduced rate of healing and regeneration of the bone and cartilage. It is expected that significant migration of the bioactive glass material occurs away from the right osteochondral defects post-surgery while minimal migration of the bioactive glass material occurs away from the left osteochondral defects post-surgery.

### Example 5

### Preparation of a Bone Repair Composition Paste

A carrier solution is prepared by dissolving 27 weight percent of Pluronic® F127 (Poloxamer 407) powder and 1 weight percent CaCl₂ in 72 weight percent sterile water at 5° C. The composition is then prepared by mixing 30 weight percent of the solution with 70 weight percent bioactive glass at 5° C. At 25° C., the composition has a paste-like consistency. If there is any concern that the bioactive glass is no longer uniformly suspended in the composition, the composition is cooled to 5° C., placed on a rotating shaker, and then gradually heated to 25° C.

### Example 6

### Preparation of Poloxamer Coated Bioactive Glass Particles for Bone Repair

A Bone Repair Paste is prepared as described in Example 5. The composition is dried at 105° C. for 2-6 hours. The dried composition is then milled and sieved to separate poloxamer coated particles. Blood or BMA can then added to the coated particles in a 1 gram to 3.5 g of particles ratio to form a bone repair matrix to be placed on or near the bone gap or the bone defect.

The following data was obtained for Examples 5 and 6:

Evaluation of Dissolution in 37° C. Water Bath

| Sample Description | Dissolution Time* |
|---|---|
| CONTROL | 5 minutes |
| (Glycerin/PEG composite containing bioactive | |
| glass particles) | |
| EXAMPLE 5 | >3 hours |
| (Poloxamer 407/water composite containing | |
| bioactive glass particles) | |
| EXAMPLE 6 | >3 hours |
| (Poloxamer 407 coated bioactive glass particles | |
| hydrated with citrated bovine blood) | |

*Time required for the sample to fall apart in water
Note:
Samples were formed into 1.5 g spheres before being placed into the water bath

The following data was obtained for Examples 5 and 6:

Evaluation of Thermoreversible Behavior

| | Temperature of | | Δ |
|---|---|---|---|
| | Sample Before | Compressive | Compressive |
| Sample Description | Evaluation | Strength (N) | Strength* |
| CONTROL | 25° C. | 2.441 ± 0.32 | −0.77 |
| (Glycerin/PEG composite | 37° C. | 1.675 ± 0.76 | |
| containing bioactive glass | | | |
| particles) | | | |
| EXAMPLE # | 25° C. | 1.887 ± 0.22 | 0.42 |
| (Poloxamer 407/water | 37° C. | 2.310 ± 0.63 | |
| composite containing | | | |
| bioactive glass particles) | | | |
| EXAMPLE ## | 25° C. | 2.328 ± 0.41 | 1.19 |
| (Poloxamer 407 coated | 37° C. | 3.517 ± 0.22 | |
| bioactive glass particles | | | |
| hydrated with citrated | | | |
| bovine blood) | | | |

NOTE:
The composites contained the same weight percent of bioactive glass and were
formed into cylinders (D = 8.5 mm, H = 12 mm) for the compression test.
*Change in compressive strength determined by 37° C.-25° C. Positive change indicates thermoreversible behavior

Throughout this specification various indications have been given as to preferred and alternative embodiments of the invention. However, the foregoing detailed description is to be regarded as illustrative rather than limiting and the invention is not limited to any one of the provided embodiments. It should be understood that it is the appended claims, including all equivalents, are intended to define the spirit and scope of this invention.

1. A bone repair composition comprising a bone repair material suspended in a mixture of non-random poly(oxyalkylene) block copolymer and water exhibiting reverse phase behavior when the temperature increases from room temperature to body temperature.

2. The bone repair composition of claim 1, wherein the mixture of poly(oxyalkylene) block copolymer and water is more viscous at body temperature than at room temperature.

3. The bone repair composition of claim 1, wherein the poly(oxyalkylene) block copolymer is a poloxamer.

4. The bone repair composition of claim 1, wherein the poly(oxyalkylene) block copolymer is poloxamer 407.

5. The bone repair composition of claim 1, wherein the weight ratio of the poly(oxyalkylene) block copolymer is 10%-90% relative to the weight of the bone repair composition.

6. The bone repair composition of claim 1, wherein the weight ratio of the poly(oxyalkylene) block copolymer is 10%-20% relative to the weight of the bone repair composition.

7. The bone repair composition of claim 1, wherein the weight ratio of the poly(oxyalkylene) block copolymer is 20%-30% relative to the weight of the bone repair composition.

8. The bone repair composition of claim 1, wherein the weight ratio of the poly(oxyalkylene) block copolymer is 30%-40% relative to the weight of the bone repair composition.

9. The bone repair composition of claim 1, wherein the weight ratio of the poly(oxyalkylene) block copolymer is 40%-50% relative to the weight of the bone repair composition.

10. The bone repair composition of claim 1, wherein the weight ratio of the poly(oxyalkylene) block copolymer is 50%-60% relative to the weight of the bone repair composition.

11. The bone repair composition of claim 1, wherein the weight ratio of the poly(oxyalkylene) block copolymer is 60%-70% relative to the weight of the bone repair composition.

12. The bone repair composition of claim 1, wherein the weight ratio of the poly(oxyalkylene) block copolymer is 70%-80% relative to the weight of the bone repair composition.

13. The bone repair composition of claim 1, wherein the weight ratio of the poly(oxyalkylene) block copolymer is 80%-90% relative to the weight of the bone repair composition.

14. The bone repair composition of claim 1, wherein the weight ratio of the poly(oxyalkylene) block copolymer is about 20% relative to the weight of the bone repair composition.

15. The bone repair composition of claim 1, wherein the weight ratio of the poly(oxyalkylene) block copolymer is about 30% relative to the weight of the bone repair composition.

16. The bone repair composition of claim 1, wherein the bone repair material is a composition comprising calcium salt and silica, wherein the silica is in the form of an inorganic silicate that is adsorbed onto the surface of the calcium salt, wherein the silica is not incorporated into the structure of the calcium salt, and wherein the composition is bioactive.

17. The bone repair composition of claim 16, wherein the calcium salt is selected from the group consisting of calcium carbonate, calcium borate, calcium sulfate, calcium phosphate, and beta calcium triphosphate.

18-21. (canceled)

22. The bone repair composition of claim 16, wherein the composition is osteoinductive.

23. The bone repair composition of claim 16, wherein a sufficient quantity of silica is present to reduce the resorption rate of calcium.

24. The bone repair composition of claim 17, wherein the silica is effective to reduce the resorption rate of calcium sulfate.

25. The bone repair composition of claim 17, wherein the silica is effective to reduce the resorption rate of beta calcium triphosphate.

26. The bone repair composition of claim 16, wherein the adsorbed silica forms a thin layer.

27. The bone repair composition of claim 26, wherein the thin layer of adsorbed silica is effective to reduce the rate of adsorption of calcium.

28. The bone repair composition of claim 17, wherein the adsorbed silica forms a thin layer effective to reduce the rate of adsorption of calcium sulfate.

29. The bone repair composition of claim 17, wherein the adsorbed silica forms a thin layer effective to reduce the rate of adsorption of calcium sulfate.

30. The bone repair composition of claim 16, wherein the calcium and silica are effective to stimulate osteoblast differentiation and osteoblast proliferation.

31. The bone repair composition of claim 16, wherein the ratio of silica and the composition is from 0.01 wt % to 50 wt %.

32. The bone repair composition of claim 16, wherein the ratio of silica and the composition is from 1 wt % to 5 wt %.

33. The bone repair composition of claim 16, wherein the inorganic silicate is substituted with a functional group.

34. The bone repair composition of claim 1, wherein the bone repair material is bioactive glass.

35. The bone repair composition of claim 34, wherein the bioactive glass is in the form of a particle.

36. The bone repair composition of claim 34, wherein the bioactive glass comprises SiO₂.

37. The bone repair composition of claim 34, wherein the bioactive glass comprises P₂O₅.

38. The bone repair composition of claim 34, wherein the bioactive glass comprises B₂O₃.

39. The bone repair composition of claim 34, wherein the bioactive glass comprises SiO₂ and P₂O₅.

40. The bone repair composition of claim 34, wherein the bioactive glass comprises SiO₂ and B₂O₃.

41. The bone repair composition of claim 34, wherein the bioactive glass comprises 40-60% SiO₂, 10-20% CaO, 0-4% P₂O₅, and 19-30% NaO.

42. The bone repair composition of claim 35, wherein the bioactive glass further comprises a carrier selected from the group consisting of hydroxyapatite and tricalcium phosphate.

43. The bone repair composition of claim 35, wherein the bioactive glass is in the form of particles having a diameter of between about 1 micrometer and about 2,000 micrometers.

44. The bone repair composition of claim 34, wherein the bioactive glass has a substantially spherical shape.

45. The bone repair composition of claim 34, wherein the bioactive glass is a microsphere.

46. The bone repair composition of claim 1, wherein the bone repair material is a bimodal bioactive glass composition comprising
large bioactive glass particles having a substantially spherical shape and a mean diameter of between about 90 micrometers and about 2000 micrometers; and
small bioactive glass particles having a substantially spherical shape and a mean diameter of between about 10 micrometers and about 500 micrometers.

47. The bone repair composition of claim 46, wherein the large bioactive glass particles have a mean diameter of between about 90 micrometers and about 200 micrometers.

48. The bone repair composition of claim 46, wherein the large bioactive glass particles have a mean diameter of between about 200 micrometers and about 400 micrometers.

49. The bone repair composition of claim 46, wherein the large bioactive glass particles have a mean diameter of between about 300 micrometers and about 500 micrometers.

50. The bone repair composition of claim 46, wherein the large bioactive glass particles have a mean diameter of between about 400 micrometers and about 600 micrometers.

51. The bone repair composition of claim 46, wherein the large bioactive glass particles have a mean diameter of between about 500 micrometers and about 700 micrometers.

52. The bone repair composition of claim 46, wherein the large bioactive glass particles have a mean diameter of between about 600 micrometers and about 800 micrometers.

53. The bone repair composition of claim 46, wherein the large bioactive glass particles have a mean diameter of between about 700 micrometers and about 900 micrometers.

54. The bone repair composition of claim 46, wherein the large bioactive glass particles have a mean diameter of between about 800 micrometers and about 1000 micrometers.

55. The bone repair composition of claim 46, wherein the large bioactive glass particles have a mean diameter of between about 900 micrometers and about 1100 micrometers.

56. The bone repair composition of claim 46, wherein the large bioactive glass particles have a mean diameter of between about 1000 micrometers and about 1200 micrometers.

57. The bone repair composition of claim 46, wherein the large bioactive glass particles have a mean diameter of between about 1100 micrometers and about 1300 micrometers.

58. The bone repair composition of claim 46, wherein the large bioactive glass particles have a mean diameter of between about 1200 micrometers and about 1400 micrometers.

59. The bone repair composition of claim 46, wherein the large bioactive glass particles have a mean diameter of between about 1300 micrometers and about 1500 micrometers.

60. The bone repair composition of claim 46, wherein the large bioactive glass particles have a mean diameter of between about 1400 micrometers and about 1600 micrometers.

61. The bone repair composition of claim 46, wherein the large bioactive glass particles have a mean diameter of between about 1500 micrometers and about 1700 micrometers.

62. The bone repair composition of claim 46, wherein the large bioactive glass particles have a mean diameter of between about 1600 micrometers and about 1800 micrometers.

63. The bone repair composition of claim 46, wherein the large bioactive glass particles have a mean diameter of between about 1700 micrometers and about 1900 micrometers.

64. The bone repair composition of claim 46, wherein the large bioactive glass particles have a mean diameter of between about 1800 micrometers and about 2000 micrometers.

65. The bone repair composition of claim 46, wherein the small bioactive glass particles have a mean diameter of between about 10 micrometers and about 30 micrometers.

66. The bone repair composition of claim 46, wherein the small bioactive glass particles have a mean diameter of between about 30 micrometers and about 50 micrometers.

67. The bone repair composition of claim 46, wherein the small bioactive glass particles have a mean diameter of between about 50 micrometers and about 70 micrometers.

68. The bone repair composition of claim 46, wherein the small bioactive glass particles have a mean diameter of between about 70 micrometers and about 90 micrometers.

69. The bone repair composition of claim 46, wherein the small bioactive glass particles have a mean diameter of between about 90 micrometers and about 110 micrometers.

70. The bone repair composition of claim 46, wherein the small bioactive glass particles have a mean diameter of between about 110 micrometers and about 130 micrometers.

71. The bone repair composition of claim 46, wherein the small bioactive glass particles have a mean diameter of between about 130 micrometers and about 150 micrometers.

72. The bone repair composition of claim 46, wherein the small bioactive glass particles have a mean diameter of between about 150 micrometers and about 170 micrometers.

73. The bone repair composition of claim 46, wherein the small bioactive glass particles have a mean diameter of between about 171 micrometers and about 190 micrometers.

74. The bone repair composition of claim 46, wherein the small bioactive glass particles have a mean diameter of between about 190 micrometers and about 200 micrometers.

75. The bone repair composition of claim 46, wherein the large bioactive glass particles do not comprise borate ions and the small bioactive glass particles do not comprise borate ions.

76. The bone repair composition of claim 46, wherein the large bioactive glass particles are microspheres.

77. The bone repair composition of claim 46, wherein the small bioactive glass particles are microspheres.

78. The bone repair composition of claim 76, wherein the microspheres are produced by flame volatilization of bioactive glass particles.

79. The bone repair composition of claim 77, wherein the microspheres are produced by flame volatilization of bioactive glass particles.

80. The bone repair composition of claim 1, wherein the bone repair material is one or more particles of bioactive glass coated with a glycosaminoglycan, wherein the glycosaminoglycan is bound to the bioactive glass.

81. The bone repair composition of claim 80, wherein the glycosaminoglycan is bound to the bioactive glass by means of an ionic bond.

82. The bone repair composition of claim 80, wherein the glycosaminoglycan is bound to the bioactive glass by means of a covalent bond.

83. The bone repair composition of claim 80, wherein the glycosaminoglycan is selected from the group consisting of heparin, heparan sulfate, chondroitin sulfate, dermatan sulfate, keratan sulfate, and hyaluronic acid.

84-88. (canceled)

89. The bone repair composition of claim 1, wherein the bone repair material comprises:
an autograft bone particle; and
a bioactive glass particle.

90. The bone repair composition of claim 89, wherein the bioactive glass particle comprises SiO₂.

91. The bone repair composition of claim 89, wherein the bioactive glass particle comprises P₂O₅, PO₃, or PO₄.

92. The bone repair composition of claim 89, wherein the bioactive glass particle comprises B₂O₃.

93. The bone repair composition of claim 89, wherein the bioactive glass particle comprises 40-60% SiO₂, 10-20% CaO, 0-4% P₂O₅, and 19-30% NaO.

94. The bone repair composition of claim 89, wherein the bioactive glass particle further comprises a carrier selected from the group consisting of hydroxyapatite and tricalcium phosphate.

95. The bone repair composition of claim 89, wherein the bioactive glass particle has a diameter of between about 1 micrometer and about 2,000 micrometers.

96. The bone repair composition of claim 89, wherein the autograft bone particle and the bioactive glass particle are pretreated in a solution comprising one or more of blood, bone marrow aspirate, bone-morphogenetic proteins, platelet-rich plasma, and osteogenic proteins.

97. The bone repair composition of claim 89, wherein the bioactive glass particle does not include any substantial amount of polymer.

98. A method for treating a bone having a bone defect comprising
contacting the bone at or near the site of the bone defect with the bone repair composition of claim 1.

99. A method for treating a bone having a bone defect comprising
placing the bone repair composition of claim 1 at a site of a bone gap or a bone defect.

100. A method for treating a bone having a bone defect comprising
placing the bone repair composition of claim 1 within a bone gap or a bone defect.

101. A bone repair composition for treating bone defects comprising bioactive glass particles with a non-random poloxamer coating.

102. A putty or paste including the bone repair composition of claim 101 mixed with water, saline, blood, or BMA.
